SEQUENCE LISTING (2 sequences)

SEQ ID NO: 1 - DNA, 3315 bp, Homo sapiens
atgtcctttcgggcagccaggctcagcatgaggaacagaaggaatgacactctggacagcacccggaccctgtactccagcgcgtctcggagcacagacttgtcttacagtgaaagcgacttggtgaattttattcaagcaaattttaagaaacgagaatgtgtcttctttatcaaagattccaaggccacggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacatcggggaggtggtgagagataacaccatcagcaggagttcagaggagaatattgtggccattggcatagcagcttggggcatggtctccaaccgggacaccctcatcaggaattgcgatgctgagggctattttttagcccagtaccttatggatgacttcacaagagatccactgtatatcctggacaacaaccacacacatttgctgctcgtggacaatggctgtcatggacatcccactgtcgaagcaaagctccggaatcagctagagaagtatatctctgagcgcactattcaagattccaactatggtggcaagatccccattgtgtgttttgcccaaggaggtggaaaagagactttgaaagccatcaatacctccatcaaaaataaaattccttgtgtggtggtggaaggctcgggccagatcgctgatgtgatcgctagcctggtggaggtggaggatgccctgacatcttctgccgtcaaggagaagctggtgcgctttttaccccgcacggtgtcccggctgcctgaggaggagactgagagttggatcaaatggctcaaagaaattctcgaatgttctcacctattaacagttattaaaatggaagaagctggggatgaaattgtgagcaatgccatctcctacgctctatacaaagccttcagcaccagtgagcaagacaaggataactggaatgggcagctgaagcttctgctggagtggaaccagctggacttagccaatgatgagattttcaccaatgaccgccgatgggagtctgctgaccttcaagaagtcatgtttacggctctcataaaggacagacccaagtttgtccgcctctttctggagaatggcttgaacctacggaagtttctcacccatgatgtcctcactgaactcttctccaaccacttcagcacgcttgtgtaccggaatctgcagatcgccaagaattcctataatgatgccctcctcacgtttgtctggaaactggttgcgaacttccgaagaggcttccggaaggaagacagaaatggccgggacgagatggacatagaactccacgacgtgtctcctattactcggcaccccctgcaagctctcttcatctgggccattcttcagaataagaaggaactctccaaagtcatttgggagcagaccaggggctgcactctggcagccctgggagccagcaagcttctgaagactctggccaaagtgaagaacgacatcaatgctgctggggagtccgaggagctggctaatgagtacgagacccgggctgttgagctgttcactgagtgttacagcagcgatgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggaagaaacctgtcgacaagcacaagaagctgctttggtactatgtggcgttcttcacctcccccttcgtggtcttctcctggaatgtggtcttctacatcgccttcctcctgctgtttgcctacgtgctgctcatggatttccattcggtgccacacccccccgagctggtcctgtactcgctggtctttgtcctcttctgtgatgaagtgagacagtggtacgtaaatggggtgaattattttactgacctgtggaatgtgatggacacgctggggcttttttacttcatagcaggaattgtatttcggctccactcttctaataaaagctctttgtattctggacgagtcattttctgtctggactacattattttcactctaagattgatccacatttttactgtaagcagaaacttaggacccaagattataatgctgcagaggatgctgatcgatgtgttcttcttcctgttcctctttgcggtgtggatggtggcctttggcgtggccaggcaagggatccttaggcagaatgagcagcgctggaggtggatattccgttcggtcatctacgagccctacctggccatgttcggccaggtgcccagtgacgtggatggtaccacgtatgactttgcccactgcaccttcactgggaatgagtccaagccactgtgtgtggagctggatgagcacaacctgccccggttccccgagtggatcaccatccccctggtgtgcatctacatgttatccaccaacatcctgctggtcaacctgctggtcgccatgtttggctacacggtgggcaccgtccaggagaacaatgaccaggtctggaagttccagaggtacttcctggtgcaggagtactgcagccgcctcaatatccccttccccttcatcgtcttcgcttacttctacatggtggtgaagaagtgcttcaagtgttgctgcaaggagaaaaacatggagtcttctgtctgctgtttcaaaaatgaagacaatgagactctggcatgggagggtgtcatgaaggaaaactaccttgtcaagatcaacacaaaagccaacgacacctcagaggaaatgaggcatcgatttagacaactggatacaaagcttaatgatctcaagggtcttctgaaagagattgctaataaaatcaaataa

SEQ ID NO: 2 - DNA, 3318 bp, Homo sapiens
atgagcttcagagcagccaggctgagcatgcggaaccggagaaacgacaccctggacagcaccagaaccctgtacagcagcgccagcagaagcaccgatctgagctacagcgagagcgacctggtgaacttcatccaggccaacttcaagaagcgggagtgcgtgttcttcatcaaggacagcaaggccaccgagaatgtgtgcaagtgcggctacgcccagagccagcacatggagggcacccagatcaaccagagcgagaagtggaactacaagaagcacaccaaggagttccctaccgacgccttcggcgacatccagttcgagaccctgggcaagaagggcaagtacatccggctgagctgcgacaccgacgccgagatcctgtacgagctgctgacccagcactggcacctgaaaacccccaacctggtgatcagcgtgaccggcggagccaagaatttcgccctgaagccccgcatgcggaagatcttctccaggctgatctacatcgcccagagcaagggcgcctggatcctgaccggcggcacccactacggcctgatgaagtacatcggcgaagttgtgcgggacaacaccatcagcaggagcagcgaggagaacatcgtggccatcggcatcgccgcctggggcatggtgtccaacagggacaccctgatcagaaactgcgacgccgagggctacttcctggcccagtacctgatggacgacttcaccagggaccccctgtacatcctggacaacaaccacacccacctgctgctcgtggataacggctgccacggccaccctaccgtggaggccaagctgagaaaccagctggagaagtacatctccgagcggaccatccaggatagcaactacggcggcaagatccccatcgtgtgtttcgcccagggcggaggaaaggagaccctgaaggccatcaacaccagcatcaagaacaagatcccctgcgtggtggtggagggcagcggccagatcgccgacgtgatcgccagcctggtggaagtggaggacgccctgaccagcagcgccgtgaaggagaagcttgtgcggttcctgcctagaaccgtgtccagactgcctgaggaggagaccgagagctggatcaagtggctgaaggagatcctggagtgcagccacctgctgaccgtgatcaagatggaggaggccggcgacgagatcgtgtccaacgccatcagctacgccctgtacaaggccttcagcaccagcgagcaggacaaggacaactggaacggccagctgaagctgctgctggagtggaatcagctggacctggccaacgacgagatcttcaccaacgaccggagatgggagagcgccgatctgcaagaagtgatgttcaccgccctgatcaaggaccggcccaagtttgtgaggctgttcctggagaacggcctgaacctgcggaagttcctgacccacgacgtgctgaccgagctgttcagcaaccacttcagcaccctggtgtaccggaatctgcagatcgccaagaacagctacaacgacgccctgctgacctttgtgtggaaactggtggccaacttccggaggggcttcaggaaggaggaccggaacggcagagatgagatggacatcgagctgcacgatgtgagccccatcaccagacaccccctgcaggccctgttcatctgggccatcctgcagaacaagaaggagctgagcaaagtgatctgggagcagaccagaggctgcaccctggccgcactcggcgccagcaagctgctgaaaaccctggccaaagtgaagaacgacatcaatgccgccggagagagcgaggagctggccaatgagtacgagaccagggccgtggagctgttcaccgagtgctacagcagcgatgaggatctggccgagcagctgctggtgtacagctgcgaggcctggggcggcagcaactgcctggagctggccgtggaagccaccgatcagcacttcatcgcccagcctggcgtgcagaacttcctgagcaagcagtggtacggcgagatcagccgggacaccaagaactggaagatcatcctgtgcctgttcatcatccctcttgtgggctgcggcttcgtgtccttcaggaagaagcccgtggacaagcacaagaagctgctgtggtactacgtggccttcttcaccagccccttcgtggtgttcagctggaacgtggtgttttacatcgcctttctgctgctgttcgcctacgtgctgctgatggacttccacagcgtgccccacccccccgagctggtgctgtacagcctggtgttcgtgctgttctgcgacgaagtgcgccagtggtacgtgaacggcgtgaactacttcaccgacctgtggaacgtgatggataccctgggcctgttctactttatcgccggcatcgtgttcagactgcacagcagcaacaagagcagcctgtacagcggcagagtgatcttctgcctggactacatcatcttcaccctgcgcctgatccacatcttcaccgtgtcccggaacctgggccccaagatcatcatgctgcagcggatgctgatcgacgtgttcttcttcctgttcctgttcgctgtgtggatggtggccttcggcgtggccagacagggcatcctgaggcagaatgagcagcggtggcggtggatcttccggagcgtgatctacgagccctacctggccatgttcggccaggtgcccagcgacgtggacggcaccacctacgacttcgcccactgcaccttcaccggcaacgagagcaagcccctgtgcgtggagctggacgagcacaacctgcccagattccccgagtggatcaccatccccctggtgtgtatctacatgctgagcaccaacatcctgctggtgaacctgctcgtggccatgtttggctacacagtgggcaccgtgcaggagaacaacgaccaagtgtggaagttccagcggtacttcctggtgcaggagtactgcagcaggctgaacatccccttccccttcatcgtgtttgcctacttctacatggtggtgaagaagtgcttcaagtgctgctgcaaggaaaagaacatggagagcagcgtgtgttgcttcaagaacgaggacaatgagaccctggcctgggagggcgtgatgaaggagaactacctggtgaagatcaacaccaaggccaacgacacctccgaggagatgcggcacagattcagacagctcgacaccaagctgaacgacctgaagggcctgctgaaggaaatcgccaacaagatcaagtgatga

(19) United States
(12) Patent Application Publication
(10) Pub. No.: US 2007/0259354 A1
(43) Pub. Date: Nov. 8, 2007

(54) OPTIMIZED TRPM8 NUCLEIC ACID SEQUENCES AND THEIR USE IN CELL BASED ASSAYS AND TEST KITS TO IDENTIFY TRPM8 MODULATORS

(75) Inventors: Guy Servant, San Diego, CA (US); Paul Brust, San Diego, CA (US); Bryan Moyer, San Diego, CA (US); Min Lu, San Diego, CA (US); Fernando Echeverri, Chula Vista, CA (US); David Dahan, Oceanside, CA (US); Mark Zoller, La Jolla, CA (US); Mark Williams, Carlsbad, CA (US); Rachel Kimmich, Carlsbad, CA (US); Poonit Kamdar, San Diego, CA (US); Tanya Ditschun, San Diego, CA (US); Andrew Patron, San Marcos, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Senomyx, Inc., San Diego, CA (US)

(21) Appl. No.: 11/536,394

(22) Filed: Sep. 28, 2006

Related U.S. Application Data
(60) Provisional application No. 60/724,776, filed on Oct. 11, 2005.
(60) Provisional application No. 60/724,777, filed on Oct. 11, 2005.

(51) Int. Cl. C12Q 1/68; C07H 21/02; C07H 21/04; C12N 15/85; C12N 5/10

(52) U.S. Cl. 435/6; 435/320.1; 435/358; 435/363; 435/365; 435/366; 435/367; 536/23.1; 536/24.1

(57) ABSTRACT

Modified human TRPM8 nucleic acid sequences which are efficiently expressed in human cells and cell-based assays and test kits containing same are provided. These assays identify TRPM8 modulators using cells that express a modified human TRPM8 nucleic acid sequence according to the invention, wherein said sequence has been modified relative to a wild-type human TRPM8 nucleic acid sequence in order to optimize ion channel expression in desired cells. Assays using these modified TRPM8 sequences have been shown to identify compounds that modulate the human TRPM8 ion channel better or comparably to known coolants such as menthol and icilin.

### RELATED APPLICATIONS

This application claims priority to and incorporates by reference U.S. provisional application Ser. No. 60/724,776 and 60/724,777 both filed on Oct. 11, 2005.

### FIELD OF THE INVENTION

The present invention relates to TRPM8 nucleic acid sequences that are modified relative to the native (wild-type) human TRPM8 nucleic acid sequence in order to enhance the expression thereof in desired cells, preferably primate cells and most preferably human cells.

Also, the invention provides cell-based assays, preferably electrophysiological and fluorimetric calcium or sodium imaging assays, and test kits for use therein that identify human TRPM8 modulatory compounds, preferably compounds that elicit a cooling sensation in human subjects approximate to the known cooling compounds menthol or icilin and/or TRPM8 modulators which potentiate the cooling sensation elicited by menthol or icilin using the subject modified TRPM8 nucleic acid sequences. The subject cell-based assays preferably use cells which express a modified human TRPM8 nucleic acid sequence which is mutated to optimize expression in recombinant host cells, preferably human cells such as HEK-293 cells. Preferably the introduced mutations do not or substantially do not alter the sequence of the polypeptide encoded by said modified human TRPM8 nucleic acid sequence relative to the native human TRPM8 nucleic acid sequence.

### BACKGROUND OF THE INVENTION

This invention relates to assays that use modified TRPM8 nucleic acid sequences for identifying novel cooling agents. Prior to the present invention, nucleic acid sequences encoding rodent and human TRPM8 nucleic acid sequences had been reported. Additionally, it has been reported that TRPM8 is a member of the TRP ion channel family which is involved in the sensation of cool to cold temperatures as well as sensation to cooling agents such as menthol and icilin. TRPM8 is a non-selective cation channel that increases its permeability to sodium or calcium upon stimulation with cold temperatures, menthol, icilin or derivatives thereof. Still further the use of native (unmodified) TRPM8 nucleic acid sequences for identifying TRPM8 modulators has been reported.

However, notwithstanding the foregoing, improved assays and test kits for identifying compounds that modulate the human TRPM8 channel are needed. In particular, assays that identify novel compounds which modulate the human TRPM8 channel at least comparably to menthol or icilin are needed. These compounds have potential application in foods, beverages, medicinals and other compositions wherein a cooling sensation is desired.

### OBJECTS OF THE INVENTION

It is an object of the invention to provide novel mutated TRPM8 nucleic acid sequences which are efficiently expressed in desired host cells, preferably human cells such as HEK-293 cells and which upon expression yield a TRPM8 ion channel polypeptide suitable for identifying TRPM8 modulators, i.e., agonists, antagonists, and enhancers that modulate cooling sensation in humans.

More particularly, it is an object of the invention to provide novel human TRPM8 nucleic acid sequences which contain mutations relative to the native sequence which are engineered to optimize expression in human cells such as HEK-293 cells wherein such mutations do not substantially alter the binding and/or functional properties of the resultant TRPM8 channel polypeptide, e.g., conservative amino acid substitutions. For example such mutations may remove one or more of the following: (i) putative human internal TATA-boxes, (ii) chi sites (iii) ribosomal entry sites, (iv) ARE, INS, or CRS sequence elements and (v) cryptic splice donor and acceptor sites. Additionally, such mutations may replace one or more codons with host cell preferred codons, particularly human preferred codons.

Still more preferably it is an object of the invention to provide the TRPM8 nucleic acid sequence contained in SEQ ID NO:2 and variants thereof.

It is another object of the invention to provide novel cell-based assays for identifying compounds that modulate the human TRPM8 ion channel.

More particularly, it is an object of the invention to provide cell-based assays for identifying compounds that modulate the human TRPM8 ion channel using test cells which express a mutated human TRPM8 nucleic acid sequence according to the invention that comprises mutations which are engineered to optimize TRPM8 expression in recombinant host cells, preferably mammalian, and most preferably human cells.

Even more particularly it is an object of the invention to provide cell-based assays for identifying compounds that modulate the activity of human TRPM8 in human cells that express a modified human TRPM8 nucleic acid sequence, i.e., possesses a different sequence than the previously reported naturally occurring human TRPM8 nucleic acid sequence, wherein such modified sequence contains mutations that enhance TRPM8 expression in human cells and further when such mutations preferably do not alter the TRPM8 protein sequence. Particularly, such mutations may remove one or more of the following: (i) putative human putative internal TATA-boxes, (ii) chi-sites, (iii) ribosomal entry sites, (iii) AT-rich or GC-rich sequence stretches, (iv) ARE, INS or CRS sequence elements and (v) cryptic splice donor and acceptor sites. Additionally, such mutations may replace one or more codons with host cell preferred codons, particularly human preferred codons.

Still more preferably, it is an object of the invention to provide cell-based assays for identifying human TRPM8 modulatory compounds that use test cells that express the mutated human TRPM8 nucleic acid sequence contained in SEQ ID NO: 2 or a variant thereof.

Even more preferably, the cell-based assays provided herein will monitor TRPM8 activity using fluorescent calcium sensitive dyes, membrane potential dyes or sodium-sensitive dyes.

Alternatively, the cell-based assays provided herein will monitor TRPM8 activity by electrophysiological methods, i.e., by patch clamping or two-electrode voltage clamping using oocytes that express a modified TRPM8 nucleic acid sequence according to the invention.

Still alternatively, the invention provides assays wherein TRPM8 activity may be detected by ion flux, e.g., radiolabeled-ion flux assays or by use of atomic spectroscope detector methods that utilize a modified TRPM8 nucleic acid sequence according to the invention.

Most preferably, the cell-based assays provided herein utilizing a modified TRPM8 nucleic acid sequence according to the invention will use a high-throughput screening platform that facilitates the screening of thousands or even millions of different putative cooling compounds wherein TRPM8 activity is monitored using calcium sensitive dyes, membrane potential dyes or sodium sensitive dyes, electrophysiologically by patch clamping or two-electrode voltage clamping, or by ion flux assays that use radiolabels or atomic absorption spectroscope detection methods.

Also, it is an object of the invention to provide novel test kits for identifying compounds that modulate human TRPM8 that comprise (i) a test cell that expresses an altered or mutated human TRPM8 nucleic acid sequence according to the invention that encodes a polypeptide identical or substantially identical to wild-type (naturally occurring) human TRPM8, which has been modified relative to the wild-type human TRPM8 nucleic acid sequence to optimize expression in recombinant mammalian cells, preferably human cells and (ii) a detection system that comprises a means for measuring TRPM8 activity, e.g., a calcium sensitive, membrane potential or sodium sensitive dye; an electrophysiological means for identifying compounds that modulate the activity of human TRPM8, or a means for detecting TRPM8-mediated ion flux, e.g., a radiolabeled ion or atomic absorption spectroscope detection means.

### BRIEF DESCRIPTION OF THE INVENTION

The present invention relates to novel mutated TRPM8 nucleic acid sequences which contain mutations that are engineered to optimize expression in desired cells, i.e., human cells such as HEK-293 cells and the use of these sequences and cells containing in assays that use a novel mutated TRPM8 nucleic acid sequence according to the invention for identifying TRPM8 modulatory compounds, preferably compounds that function as cooling agents themselves and/or compounds which enhance the cooling effect of other cooling compounds, e.g., cooling agents such as menthol, icilin, and their derivatives.

As noted previously, TRPM8 is a non-selective cation channel in the TRP ion channel family that increases its permeability to sodium or calcium upon stimulation with cold temperatures or compounds that elicit a cooling effect such as menthol, icilin and derivatives thereof. Therefore, cells which transiently or stably express TRPM8 are useful in screens, e.g., high-throughput platform screens to identify and quantify the effects of TRPM8 modulators.

More particularly, the present invention relates to modified TRPM8 nucleic acid sequences and cell-based assays that use test cells which express these mutated or altered human TRPM8 nucleic acid sequences that have been engineered to optimize expression in mammalian cells, preferably human cells. Such optimized sequence will preferably retain the identical amino acid sequence as the wild-type human TRPM8 polypeptide or will only comprise inconsequential modifications. For example, a modified TRPM8 a sequence according to the invention may possess at least 85% sequence identity to native human TRPM8 polypeptide, more preferably at least 90-95% sequence identity, and still more preferably at least 96-99% sequence identity therewith.

The present invention exemplifies a particular modified TRPM8 nucleic acid sequence and cells that express said modified human TRPM8 nucleic acid sequence that encodes a polypeptide identical to the native human TRPM8 polypeptide wherein said modified TRPM8 nucleic acid sequence is contained in SEQ ID NO. 2 This sequence has been modified relative to the native TRPM8 nucleic acid sequence to remove putative internal TATA-boxes, chi-sites and ribosomal entry sites; AT-rich and GC-rich sequence stretches, ARE, INS and CRS sequence elements and cryptic splice donor and acceptor sites. This sequence contained in SEQ ID NO:2 contains 601 silent nucleotide substitution mutations, and exhibits 81% nucleotide sequence identity to the reported human TRPM8 nucleic acid sequence contained in SEQ ID NO: 1 infra. Cell-based assays using this optimized TRPM8 sequence have been demonstrated to be capable of identifying compounds that are equipotent or superior to menthol at activating rat and human TRPM8.

### BRIEF DESCRIPTION OF THE FIGURES

FIG. 1 contains a sequence alignment of an optimized hTRPM8 sequence used in the assays of the present invention and the previously reported wild-type hTRPM8 sequence. The wild-type sequence is contained in SEQ ID NO: 1 and the altered sequence in SEQ ID NO:2.

FIG. 2 contains the results of fluorimetric calcium imaging experiments using HEK-293 cells that transiently express a rat TRPM8 nucleic acid sequence.

FIG. 3 contains the results of fluorimetric calcium imaging experiments using HEK-293 cells expressing rat TRPM8 which are stimulated with different cooling agents.

FIG. 4 contains the results of fluorimetric calcium imaging experiments wherein HEK-293 cells that express rat TRPM8 were stimulated with different cooling agents and reduced temperatures.

FIG. 5 contains the results of an electrophysiologic (voltage clamp) assay using oocytes that express rat TRPM8 which were stimulated with menthol and icilin.

FIG. 6 contains the results of another electrophysiologic (voltage clamp) assay wherein oocytes that express rat TRPM8 were stimulated with various compounds including known cooling agents (menthol, eucalyptol, icilin, et al.).

FIG. 7 contains the results of an electrophysiologic TRPM8 assay which revealed that menthol current/voltage (i/v) curves display outward rectification in oocytes which express rat TRPM8.

FIG. 8 contains the results of an electrophysiologic TRPM8 assay wherein rat TRPM8-expressing oocytes were stimulated with menthol at different concentrations.

FIG. 9 contains the results of an electrophysiologic assay wherein oocytes expressing rat TRPM8 were stimulated with cool temperatures.

FIG. 10 contains the results of calcium imaging experiments wherein HEK-293 clones stably expressing rat TRPM8 were stimulated with different compounds including several known cooling agents.

FIG. 11 contains the results of a calcium imaging experiment wherein a HEK-293 clone stably expressing rat TRPM8 was screened against a library of nineteen thousand compounds which identified a novel compound (SID 2346448) that is about 2-3 times more potent than menthol at activating rat TRPM8.

FIG. 12 contains the results of a calcium imaging experiment wherein HEK-293 clones stably expressing rat TRPM8 was screened against the same library of nineteen thousand compounds which identified a proprietary compound (SID 576583) that is as potent as menthol at activating rat TRPM8.

FIG. 13 contains the results of another calcium imaging experiment wherein HEK-293 clone stably expressing rat TRPM8 was screened against the same compound library which revealed the identity of another proprietary compound, (SID 3498787), which reproducibly is as potent as menthol at activating rat TRPM8.

FIG. 14 contains the results of TRPM8 calcium imaging experiments wherein HEK-293 cells expressing the modified human TRPM8 nucleic acid sequence contained in SEQ ID NO2. were stimulated with several known cooling agents (menthol, WS-3, WS-23 and icilin) as well as the compounds identified in the experiments in FIGS. 11-13.

FIG. 15 contains the results of calcium imaging experiment wherein HEK-293 clones stably expressing the modified TRPM8 nucleic acid sequence in SEQ ID NO2: were stimulated with several known cooling compounds (menthol, coolant P, WS-3, icilin).

FIG. 16 contains a table summarizing the results of dose-response experiments wherein HEK293 cells stably expressing the modified human TRPM8 nucleic acid sequence contained in SEQ ID NO2 were stimulated with known coolants as well as novel compounds identified by high throughput screening including compounds identified in the experiments in FIGS. 11-13.

FIG. 17 contains the results of an experiment wherein a compound identified as a potential cooling agent (SID 391254) using cells which express the subject modified TRPM8 nucleic acid sequence was screened for its cooling effect in human volunteers.

FIG. 18 contains the results of another experiment wherein a compound identified as a potential cooling agent (SID 10135651) was screened for its cooling effect in human volunteers.

FIG. 19 contains the results of an experiment wherein another compound identified as a potential cooling agent (SID 7292725) was screened for its cooling effect in human volunteers.

### DETAILED DESCRIPTION OF THE INVENTION AND RELEVANT TERMS

The present invention provides modified TRPM8 nucleic acid sequences and cell-based assays and test kits that express or contain such sequences that are useful to identify TRPM8 modulators. As discussed in detail infra, these cell-based assays which use cells which express a modified TRPM8 nucleic acid sequence according to the invention preferably use high throughput screening platforms to identify compounds that modulate TRPM8 activity in mammalian cells preferably human cells. These assays that use cells that express the subject modified TRPM8 nucleic acid sequences or a rodent TRPM8 will preferably be effected using fluorescent calcium sensitive dyes such as Fura2, Fluo3 or Fluo4 as well as membrane potential dyes or sodium-sensitive dyes. Alternatively, compounds that modulate TRPM8 are preferably identified by high throughput electrophysiological screens using oocytes that express the subject modified human TRPM8 nucleic acid sequence or a rodent TRPM8 by patch clamping or two electrode voltage clamping.

Still alternatively, compounds that modulate TRPM8 may be detected by ion flux assays, e.g., radiolabeled-ion flux assays or atomic absorption spectroscopic coupled ion flux assays using cells which express a modified TRPM8 nucleic acid sequence according to the invention.

The inventive modified TRPM8 nucleic acid sequences are genetically engineered to optimize expression in desired cells, preferably human cells such as HEK-293 cells and oocytes or other human cells conventionally used in screens for identifying GPCR and ion channel modulatory compounds.

TRPM8 proteins are known to form channels that have cation channel activity; in particular they exhibit calcium and sodium permeability. The protein has relatively high permeability to calcium and little selectivity among monovalent cations. Channel activity can be effectively measured, e.g., by recording ligand-induced changes in [Ca²⁺]ᵢ and measuring calcium influx using fluorescent Ca²⁺-indicator dyes and fluorimetric imaging. TRPM8 is expressed in a number of tissues, including sensory neurons, as well as prostate epithelia and a variety of tumors, e.g., other epithelial tumors. Additional tissues that may express TRPM8 or homologues include the brain and regions of the brain, such as the hypothalamus, that regulate core body temperature.

Within the TRP family, TRPM2 and TRPM7 have been electrophysiologically characterized and shown to behave as bifunctional proteins in which enzymatic activities associated with their long C-terminal domains are believed to regulate channel opening. Specifically, TRPM2 contains a Nudix motif associated with adenosine-5′-diphosphoribose (ADPR) pyrophosphatase activity and is gated by cytoplasmic ADPR and nicotinamide adenine dinucleotide (NAD) (Perraud et al., Nature 411:595-9 (2001); Sano et al., Science 293:1327-30 (2001)). TRPM7 contains a protein kinase domain that is required for channel activation (Runnels et al., Science 291:1043-7 (2001)). In contrast, TRPM8 has a significantly shorter C-terminal region and does not contain any known enzymatic domains that might be associated with channel regulation.

TRPM8 encodes a channel protein that is sensitive to temperatures that encompass all of the innocuous cool (e.g., 15 to 28° C.) and part of the noxious cold (e.g., 8 to 15° C.) range. Furthermore, it has been suggested that TRPM8 may contribute to depolarization of fibers at temperatures in the ultra-cold range (<8° C.), for example, if the channel is modified or modulated in a manner that extends its sensitivity range in vivo. Indeed, VR1 and several other members of the TRP channel family are regulated by receptors that couple to phospholipase C (PLC). In particular, the thermal activation threshold for VR1 can be markedly shifted to lower temperatures by inflammatory agents that either activate PLC signaling systems (e.g. bradykinin and nerve growth factor) or modulate the channel directly (e.g. protons and lipids) (Caterina & Julius, Annu. Rev. Neurosci. 24:487-517 (2001); Chuang et al., Nature 411:957-62 (2001)).

When applied to skin or mucous membranes, menthol produces a cooling sensation, inhibits respiratory reflexes and, at high doses, elicits a pungent or irritant effect that is accompanied by local vasodilation (Eccles, J. Pharm. Pharmacol. 46:618-30 (1994); Eccles, Appetite 34:29-35 (2000)). Most, if not all, of these physiological actions can be explained by excitation of sensory nerve endings within these tissues, but TRPM8 receptors elsewhere may also contribute to these or other effects of cooling compounds or cold stimuli.

As discussed above, the invention provides methods of screening for modulators, e.g., activators, inhibitors, stimulators, enhancers, etc., of TRPM8 nucleic acids and proteins, using the modified human TRPM8 nucleic acid sequences provided herein as well as rodent TRPM8. Such modulators can affect TRPM8 activity, e.g., by modulating TRPM8 transcription, translation, mRNA or protein stability; by altering the interaction of TRPM8 with the plasma membrane, or other molecules; or by affecting TRPM8 protein activity. Compounds are screened, e.g., using high throughput screening (HTS), to identify those compounds that can bind to and/or modulate the activity of a TRPM8 polypeptide or fragment thereof. In the present invention, TRPM8 proteins are recombinantly expressed in cells, e.g., human cells, and the modulation of TRPM8 is assayed by using any measure of ion channel function, such as measurement of the membrane potential, or measures of changes in intracellular calcium levels. Methods of assaying ion, e.g., cation, channel function include, for example, patch clamp techniques, two electrode voltage clamping, measurement of whole cell currents, and fluorescent imaging techniques that use Ca²⁺-sensitive fluorescent dyes such as Fura-2, Fluo3 or Fluo4, and ion flux assays, e.g., radiolabeled-ion flux assays or ion flux assays.

A TRPM8 agonist identified as set forth in the current application can be used for a number of different purposes. For example, a TRPM8 activator can be included as a flavoring or perfuming agent in foods, beverages, soaps, medicines, soaps, etc. They can also be used in medicaments to provide a cooling or soothing sensation. Also, the subject compounds may be used in insect repellants or other topical formulations, e.g., sunscreens, cosmetics, suntan lotions, skin ointments and the like. Also, TRPM8 modulators can also be used to treat diseases or conditions associated with TRPM8 activity, such as pain. Additionally, the invention provides kits for carrying out the herein-disclosed assays.

Definitions

The term “cold perception” or “cold sensation” as used herein is the ability to perceive or respond to cold stimuli. Such stimuli include cold or cool temperatures, e.g., temperatures less than about 30° C., and naturally occurring or synthetic compounds such as menthol (Eccles, J. Pharm. Pharmacol 46:618-630, 1994), eucalyptol, icilin (Wei & Seid, J. Pharm. Pharmacol. 35:110-112, 1983) and the like that elicit a cold sensation.

The term “pain” refers to all categories of pain, including pain that is described in terms of stimulus or nerve response, e.g., somatic pain (normal nerve response to a stimulus such as cold or menthol) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischemia, and diabetic neuropathy (see, e.g., Harrison's Principles of Internal Medicine, pp. 93-98 (Wilson et al., eds., 12th ed. 1991); Williams et al., J. of Medicinal Chem. 42:1481-1485 (1999), herein each incorporated by reference in their entirety).

“Somatic” pain, as described above, refers to a normal nerve response to a stimulus, often a noxious stimulus such as injury or illness, e.g., cold, heat, trauma, burn, infection, inflammation, or disease process such as cancer, and includes both cutaneous pain (e.g., skin, muscle or joint derived) and visceral pain (e.g., organ derived).

“Neuropathic” pain, as described above, refers to pain resulting from injury to or chronic changes in peripheral and/or central sensory pathways, where the pain often occurs or persists without an obvious noxious input.

“Cation channels” are a diverse group of proteins that regulate the flow of cations across cellular membranes. The ability of a specific cation channel to transport particular cations typically varies with the valency of the cations, as well as the specificity of the given channel for a particular cation.

“Homomeric channel” refers to a cation channel composed of identical alpha subunits, whereas “heteromeric channel” refers to a cation channel composed of two or more different types of alpha subunits. Both homomeric and heteromeric channels can include auxiliary beta subunits.

A “beta subunit” is a polypeptide monomer that is an auxiliary subunit of a cation channel composed of alpha subunits; however, beta subunits alone cannot form a channel (see, e.g., U.S. Pat. No. 5,776,734). Beta subunits are known, for example, to increase the number of channels by helping the alpha subunits reach the cell surface, change activation kinetics, and change the sensitivity of natural ligands binding to the channels. Beta subunits can be outside of the pore region and associated with alpha subunits comprising the pore region. They can also contribute to the external mouth of the pore region.

The term “authentic” or “wild-type” or “native” human TRPM8 nucleic acid sequence contained in SEQ ID NO:1.

The term “authentic” or “wild-type” or “native” human TRPM8 polypeptide refers to the polypeptide encoded by the nucleic acid sequence contained in SEQ ID NO:1.

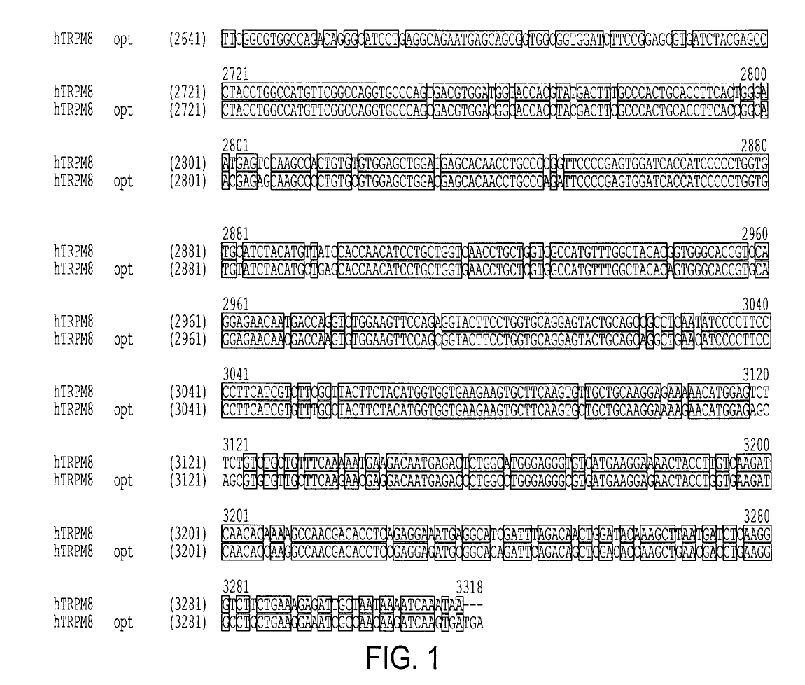

The term “modified hTRPM8 nuclear acid sequence” or “optimized hTRPM8 nucleic acid sequence” refers to a hTRPM8 nucleic acid sequence which has been genetically engineered to introduce mutations that favor expression in recombinant host cells, and most especially human cells such as HEK-293 cells. Particularly, these mutations include introducing silent mutations in the authentic hTRPM8 nuclear acid sequence as shown in SEQ ID NO:1 (FIG. 1) that remove one or more of the following: (i) TATA-boxes (ii) chi-sites, (iii) ribosomal entry sites, (iv) ARE sequence elements, (v) INS sequence elements, (vi) CRS sequence elements and/or (vii) cryptic splice donor and acceptor sites. The exemplified modified TRPM8 nucleic acid sequence contains 601 silent nucleotide modifications. Typically, modified TRPM8 nucleic acid sequences according to the invention will comprise at least 100 silent mutations, more typically at least 200-400 silent mutations, and even more typically at least 400-600 silent mutations. Exemplary appropriate silent mutations are shown in FIG. 1. Further, the sequence may be modified to introduce host cell preferred codons, particularly human host cell preferred codons. Also, the modified hTRPM8 nucleic acid sequence may be additionally modified to include non-silent mutation, e.g., conservative amino acid substitution mutations, provided that such mutations do not substantially affect the ligand binding and functional properties of the TRPM8 ion channel. An exemplary modified hTRPM8 nucleic acid sequence which is useful in assays according to the invention is contained in SEQ ID NO:2.

The term “TRPM8” protein or fragment thereof, or a nucleic acid encoding “TRPM8” or a fragment thereof refer to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by a TRPM8 nucleic acid or amino acid sequence of a TRPM8 protein, e.g., the protein encoded by SEQ ID NO:1; (2) specifically bind to antibodies, e.g., polyclonal antibodies, raised against an immunogen comprising an amino acid sequence of a TRPM8 protein or immunogenic fragments thereof, and conservatively modified variants thereof; (3) specifically hybridize under stringent hybridization conditions to an anti-sense strand corresponding to a nucleic acid sequence (SEQ ID NO:1) encoding a TRPM8 protein, and conservatively modified variants thereof; (4) have a nucleic acid sequence that has greater than about 60% sequence identity, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to a TRPM8 nucleic acid, e.g., SEQ ID NO:1 or another known TRPM8 nucleic acid sequence. The nucleic acid and amino acid sequences for rat TRPM8 have been deposited under GenBank Accession No. AY072788 and NM₁₃₄₃₇₁, see also McKemy et al., Nature 416:52-58 (2002) and SEQ ID NO:1. The nucleic acid and amino acid sequences for human TRPM8 have been deposited under GenBank Accession No. NM₀₂₄₀₈₀ and AY090109, see also Tsavaler et al., Cancer Res. 61:3760-3769, 2001; U.S. Pat. No. 6,194,152, and WO 99/09166. The nucleic acid and amino acid sequences for mouse TRPM8 have been deposited under GenBank Accession No. NM₁₃₄₂₅₂, see also Peier et al., Cell 108:705-715 (2002).

A TRPM8 polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. TRPM8 proteins typically have calcium ion channel activity, i.e., they are permeable to calcium.

By “determining the functional effect” or “determining the effect on the cell” is meant assaying the effect of a compound that increases or decreases a parameter that is indirectly or directly under the influence of a TRPM8 polypeptide e.g., functional, physical, phenotypic, and chemical effects. Such functional effects include, but are not limited to, changes in ion flux, membrane potential, current amplitude, and voltage gating, a as well as other biological effects such as changes in gene expression of TRPM8 or of any marker genes, and the like. The ion flux can include any ion that passes through the channel, e.g., calcium, and analogs thereof such as radioisotopes. Such functional effects can be measured by any means known to those skilled in the art, e.g., patch clamping, using voltage-sensitive dyes, or by measuring changes in parameters such as spectroscopic characteristics (e.g., fluorescence, absorbance, refractive index), hydrodynamic (e.g., shape), chromatographic, or solubility properties.

“Inhibitors,” “activators,” and “modulators” of TRPM8 polynucleotide and polypeptide sequences are used to refer to activating, inhibitory, or modulating molecules identified using in vitro and in vivo assays of TRPM8 polynucleotide and polypeptide sequences. Inhibitors are compounds that, e.g., bind to, partially or totally block activity, decrease, prevent, delay activation, inactivate, desensitize, or down regulate the activity or expression of TRPM8 proteins, e.g., antagonists. “Activators” are compounds that increase, open, activate, facilitate, enhance activation, sensitize, agonize, or up regulate TRPM8 protein activity. Inhibitors, activators, or modulators also include genetically modified versions of TRPM8 proteins, e.g., versions with altered activity, as well as naturally occurring and synthetic ligands, antagonists, agonists, peptides, cyclic peptides, nucleic acids, antibodies, antisense molecules, siRNA, ribozymes, small organic molecules and the like. Such assays for inhibitors and activators include, e.g., expressing TRPM8 protein in vitro, in cells, cell extracts, or cell membranes, applying putative modulator compounds, and then determining the functional effects on activity, as described above.

Samples or assays comprising TRPM8 proteins that are treated with a potential activator, inhibitor, or modulator are compared to control samples without the inhibitor, activator, or modulator to examine the extent of activation or migration modulation. Control samples (untreated with inhibitors) are assigned a relative protein activity value of 100%. Inhibition of TRPM8 is achieved when the activity value relative to the control is about 80%, preferably 50%, more preferably 25-0%. Activation of TRPM8 is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, siRNA, oligonucleotide, ribozyme, etc., to be tested for the capacity to modulate cold sensation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequences SEQ ID NO:1), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nucl. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci., USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, •-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (_{3rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., transmembrane domains, pore domains, and cytoplasmic tail domains. Domains are portions of a polypeptide that form a compact unit of the polypeptide and are typically 15 to 350 amino acids long. Exemplary domains include extracellular domains, transmembrane domains, and cytoplasmic domains. Typical domains are made up of sections of lesser organization such as stretches of .beta.-sheet and .alpha.-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include ^{32P}, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2.times. SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1.times. SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

The term antibody, as used herein, also includes antibody fragments either produced by the modification of whole antibodies, or those synthesized de novo using recombinant DNA methodologies (e.g., single chain Fv), chimeric, humanized or those identified using phage display libraries (see, e.g., McCafferty et al., Nature 348:552-554 (1990)) For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988) and Harlow & Lane, Using Antibodies, A Laboratory Manual (1999); and Goding, Monoclonal Antibodies: Principles and Practice (2d ed. 1986)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to TRPM8 protein as encoded by SEQ ID NO:1, polymorphic variants, alleles, orthologs, and conservatively modified variants, or splice variants, or portions thereof, can be selected to obtain only those polyclonal antibodies that are specifically immunoreactive with TRPM8 proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

Recombinant Expression of TRPM8

To obtain high level expression of a cloned gene, such as those cDNAs encoding TRPM8, one typically subclones TRPM8 into an expression vector that contains a strong promoter to direct transcription, a transcription/translation terminator, and if for a nucleic acid encoding a protein, a ribosome binding site for translational initiation. Suitable eukaryotic and prokaryotic promoters are well known in the art and described, e.g., in Sambrook et al., and Ausubel et al., supra. For example, bacterial expression systems for expressing the TRPM8 protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. For example, retroviral expression systems may be used in the present invention. As described infra, the subject modified hTRPM8 is preferably expressed in human cells such as HEK-293 cells which are widely used for high throughput screening.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the TRPM8-encoding nucleic acid in host cells. A typical expression cassette thus contains a promoter operably linked to the nucleic acid sequence encoding TRPM8 and signals required for efficient polyadenylation of the transcript, ribosome binding sites, and translation termination. Additional elements of the cassette may include enhancers and, if genomic DNA is used as the structural gene, introns with functional splice donor and acceptor sites. As noted previously, the exemplified modified hTRPM8 is modified to remove putative cryptic splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

The vectors used in the invention may include a regulatable promoter, e.g., tet-regulated systems and the RU-486 system (see, e.g., Gossen & Bujard, Proc. Nat'l Acad. Sci. USA 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a TRPM8 encoding sequence under the direction of the polyhedrin promoter or other strong baculovirus promoters.

The elements that are typically included in expression vectors also include a replicon that functions in the particular host cell. In the case of E. coli, the vector may contain a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods may be used to produce bacterial, mammalian, yeast or insect cell lines that express large quantities of TRPM8 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983). Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing TRPM8.

After the expression vector is introduced into the cells, the transfected cells are cultured under conditions favoring expression of TRPM8. In some instances, such TRPM8 polypeptides may be recovered from the culture using standard techniques identified below.

Assays for Modulators of TRPM8 Protein

Modulation of a TRPM8 protein, can be assessed using a variety of in vitro and in vivo assays, including cell-based models as described above. Such assays can be used to test for inhibitors and activators of TRPM8 protein or fragments thereof, and, consequently, inhibitors and activators of cold sensation. Such modulators of TRPM8 protein are useful for creating a perception of coolness, e.g., for use in medications or as flavorings, or treating disorders related to cold perception. Modulators of TRPM8 protein are tested using either recombinant or naturally occurring TRPM8.

As noted above, preferably the TRPM8 protein used in the subject cell based assays will preferably be encoded by a hTRPM8 nucleic acid sequence that has been engineered to optimize expression in specific cells, preferably human cells, and more preferably will be encoded by the modified human TRPM8 nucleic acid sequence contained in SEQ ID NO:2 or will be a rat TRPM8 polypeptide

Measurement of cold sensation phenotype of TRPM8 protein or cell expressing TRPM8 protein, either recombinant or naturally occurring, can be performed using a variety of assays, in vitro, in vivo, and ex vivo, as described herein. To identify molecules capable of modulating TRPM8, assays are performed to detect the effect of various candidate modulators on TRPM8 activity in a cell.

The channel activity of TRPM8 proteins can be assayed using a variety of assays to measure changes in ion fluxes including patch clamp techniques, measurement of whole cell currents, radiolabeled ion flux assays or a flux assay coupled to atomic absorption spectroscopy, and fluorescence assays using voltage-sensitive dyes or calcium or sodium sensitive dyes (see, e.g., Vestergarrd-Bogind et al., J. Membrane Biol. 88:67-75 (1988); Daniel et al., J. Pharmacol. Meth. 25:185-193 (1991); Hoevinsky et al., J. Membrane Biol. 137:59-70 (1994)). For example, a nucleic acid encoding a TRPM8 protein or homolog thereof can be injected into Xenopus oocytes or transfected into mammalian cells, preferably human cells such as HEK-293 cells. Channel activity can then be assessed by measuring changes in membrane polarization, i.e., changes in membrane potential.

A preferred means to obtain electrophysiological measurements is by measuring currents using patch clamp techniques, e.g., the “cell-attached” mode, the “inside-out” mode, and the “whole cell” mode (see, e.g., Ackerman et al., New Engl. J. Med. 336:1575-1595, 1997). Whole cell currents can be determined using standard methodology such as that described by Hamil et al., Pflugers. Archiv. 391:185 (1981).

Channel activity is also conveniently assessed by measuring changes in intracellular Ca²⁺ levels. Such methods are exemplified herein. For example, calcium flux can be measured by assessment of the uptake of ⁴⁵Ca²⁺ or by using fluorescent dyes such as Fura-2. In a typical microfluorimetry assay, a dye such as Fura-2, which undergoes a change in fluorescence upon binding a single Ca²⁺ ion, is loaded into the cytosol of TRPM8-expressing cells. Upon exposure to TRPM8 agonist, an increase in cytosolic calcium is reflected by a change in fluorescence of Fura-2 that occurs when calcium is bound.

The activity of TRPM8 polypeptides can in addition to these preferred methods also be assessed using a variety of other in vitro and in vivo assays to determine functional, chemical, and physical effects, e.g., measuring the binding of TRPM8 to other molecules, including peptides, small organic molecules, and lipids; measuring TRPM8 protein and/or RNA levels, or measuring other aspects of TRPM8 polypeptides, e.g., transcription levels, or physiological changes that affects TRPM8 activity. When the functional consequences are determined using intact cells or animals, one can also measure a variety of effects such as changes in cell growth or pH changes or changes in intracellular second messengers such as IP3, cGMP, or cAMP, or components or regulators of the phospholipase C signaling pathway. Such assays can be used to test for both activators and inhibitors of KCNB proteins. Modulators thus identified are useful for, e.g., many diagnostic and therapeutic applications.

In Vitro Assays

Assays to identify compounds with TRPM8 modulating activity are preferably performed in vitro. The assays herein preferably use full length TRPM8 protein or a variant thereof. This protein can optionally be fused to a heterologous protein to form a chimera. In the assays exemplified herein, cells which express the full-length TRPM8 polypeptide are used in high throughput assays are used to identify compounds that modulate cold sensation. Alternatively, purified recombinant or naturally occurring TRPM8 protein can be used in the in vitro methods of the invention. In addition to purified TRPM8 protein or fragment thereof, the recombinant or naturally occurring TRPM8 protein can be part of a cellular lysate or a cell membrane. As described below, the binding assay can be either solid state or soluble. Preferably, the protein, fragment thereof or membrane is bound to a solid support, either covalently or non-covalently. Often, the in vitro assays of the invention are ligand binding or ligand affinity assays, either non-competitive or competitive (with known extracellular ligands such as menthol). Other in vitro assays include measuring changes in spectroscopic (e.g., fluorescence, absorbance, refractive index), hydrodynamic (e.g., shape), chromatographic, or solubility properties for the protein.

Preferably, a high throughput binding assay is performed in which the TRPM8 protein is contacted with a potential modulator and incubated for a suitable amount of time. A wide variety of modulators can be used, as described below, including small organic molecules, peptides, antibodies, and TRPM8 ligand analogs. A wide variety of assays can be used to identify TRPM8-modulator binding, including labeled protein-protein binding assays, electrophoretic mobility shifts, immunoassays, enzymatic assays such as phosphorylation assays, and the like. In some cases, the binding of the candidate modulator is determined through the use of competitive binding assays, where interference with binding of a known ligand is measured in the presence of a potential modulator. Ligands for the TRPM8 family are known (e.g., menthol). Either the modulator or the known ligand is bound first, and then the competitor is added. After the TRPM8 protein is washed, interference with binding, either of the potential modulator or of the known ligand, is determined. Often, either the potential modulator or the known ligand is labeled.

In addition, high throughput functional genomics assays can also be used to identify modulators of cold sensation by identifying compounds that disrupt protein interactions between TRPM8 and other proteins to which it binds. Such assays can, e.g., monitor changes in cell surface marker expression, changes in intracellular calcium, or changes in membrane currents using either cell lines or primary cells. Typically, the cells are contacted with a cDNA or a random peptide library (encoded by nucleic acids). The cDNA library can comprise sense, antisense, full length, and truncated cDNAs. The peptide library is encoded by nucleic acids. The effect of the cDNA or peptide library on the phenotype of the cells is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the TRPM8 protein encoded by the cDNA (e.g., modified DNA contained in SEQ ID NO:2) can be isolated using a yeast two-hybrid system, mammalian two hybrid system, or phage display screen, etc. Targets so identified can be further used as bait in these assays to identify additional components that may interact with the TRPM8 channel which members are also targets for drug development (see, e.g., Fields et al., Nature 340:245 (1989); Vasavada et al., Proc. Nat'l Acad. Sci. USA 88:10686 (1991); Fearon et al., Proc. Nat'l Acad. Sci. USA 89:7958 (1992); Dang et al., Mol. Cell. Biol. 11:954 (1991); Chien et al., Proc. Nat'l Acad. Sci. USA 9578 (1991); and U.S. Pat. Nos. 5,283,173, 5,667,973, 5,468,614, 5,525,490, and 5,637,463).

Cell-Based In Vivo Assays

In another embodiment, TRPM8 protein can be expressed in a cell, and functional, e.g., physical and chemical or phenotypic, changes are assayed to identify TRPM8 modulators that modulate cold sensations. Cells expressing TRPM8 proteins can also be used in binding assays. Any suitable functional effect can be measured, as described herein. For example, changes in membrane potential, changes in intracellular calcium or sodium levels, and ligand binding are all suitable assays to identify potential modulators using a cell based system. Suitable cells for such cell based assays include both primary cells, e.g., sensory neurons from the dorsal root ganglion and cell lines that express a TRPM8 protein. The TRPM8 protein can be naturally occurring or recombinant. Also, as described above, fragments of TRPM8 proteins or chimeras with ion channel activity can be used in cell based assays. For example, a transmembrane domain of a TRPM8 protein can be fused to a cytoplasmic domain of a heterologous protein, preferably a heterologous ion channel protein. Such a chimeric protein would have ion channel activity and could be used in cell based assays of the invention. In another embodiment, a domain of the TRPM8 protein, such as the extracellular or cytoplasmic domain, is used in the cell-based assays of the invention.

In another embodiment, cellular TRPM8 polypeptide levels can be determined by measuring the level of protein or mRNA. The level of TRPM8 protein or proteins related to TRPM8 ion channel activation are measured using immunoassays such as western blotting, ELISA and the like with an antibody that selectively binds to the TRPM8 polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, TRPM8 expression can be measured using a reporter gene system. Such a system can be devised using a TRPM8 protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

In another embodiment, a functional effect related to signal transduction can be measured. An activated or inhibited TRPM8 will alter the properties of target enzymes, second messengers, channels, and other effector proteins. The examples include the activation of phospholipase C and other signaling systems. Downstream consequences can also be examined such as generation of diacyl glycerol and IP3 by phospholipase C.

Assays for TRPM8 activity include cells that are loaded with ion or voltage sensitive dyes to report receptor activity, e.g., by observing calcium influx or intracellular calcium release. Assays for determining activity of such receptors can also use known agonists and antagonists for TRPM8 receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. Radiolabeled ion flux assays or a flux assay coupled to atomic absorption spectroscopy can also be used.

Animal Models

Animal models of cold sensation also find use in screening for modulators of lymphocyte activation or migration. Similarly, transgenic animal technology including gene knockout technology, for example as a result of homologous recombination with an appropriate gene targeting vector, or gene overexpression, will result in the absence or increased expression of the TRPM8 protein. The same technology can also be applied to make knock-out cells. When desired, tissue-specific expression or knockout of the TRPM8 protein may be necessary. Transgenic animals generated by such methods find use as animal models of cold responses.

Knock-out cells and transgenic mice can be made by insertion of a marker gene or other heterologous gene into an endogenous TRPM8 gene site in the mouse genome via homologous recombination. Such mice can also be made by substituting an endogenous TRPM8 with a mutated version of the TRPM8 gene, or by mutating an endogenous TRPM8, e.g., by exposure to known mutagens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual (1988) and Teratocarcinomas and Embryonic Stem Cells: A Practical Approach (Robertson, ed., 1987).

Candidate TRPM8 Modulators

The compounds tested as modulators of TRPM8 protein can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of an TRPM8 protein. Typically, test compounds will be small organic molecules, peptides, lipids, and lipid analogs. In one embodiment, the compound is a menthol analog, either naturally occurring or synthetic.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md.).

C. Solid State and Soluble High Throughput Assays

Additionally soluble assays can be effected using a TRPM8 protein, or a cell or tissue expressing a TRPM8 protein, either naturally occurring or recombinant. Still alternatively, solid phase based in vitro assays in a high throughput format can be effected, where the TRPM8 protein or fragment thereof, such as the cytoplasmic domain, is attached to a solid phase substrate. Any one of the assays described herein can be adapted for high throughput screening, e.g., ligand binding, calcium flux, change in membrane potential, etc.

In the high throughput assays of the invention, either soluble or solid state, it is possible to screen several thousand different modulators or ligands in a single day. This methodology can be used for TRPM8 proteins in vitro, or for cell-based or membrane-based assays comprising an TRPM8 protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:6031-6040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753-759 (1996) (all describing arrays of biopolymers fixed to solid substrates). Non-chemical approaches for fixing tag binders to substrates include other common methods, such as heat, cross-linking by UV radiation, and the like.

Having described the invention supra, the following examples provide the further illustration of some preferred embodiments of the invention. These examples are provided only for purposes of illustration and should not be construed as limiting the subject invention.

### EXAMPLES

### Example 1

### Construction of a Modified hTRPM8 Nucleic Acid Sequence According to the Invention

A modified human TRPM8 nucleic acid sequence was constructed using the native hTRPM8 sequence as a template. Specifically, in order to optimize expression of hTRPM8 in recombinant host cells (preferably human cells such as HEK-293 cells) 601 silent mutations were introduced into the native hTRPM8 nucleic acid sequence resulting in a modified sequence only possessing 81% sequence identity to the parent sequence. The mutations (shown in the alignment contained in FIG. 1) were made to remove putative TATA-boxes, chi-sites and ribosomal entry sites, AT-rich or GC-rich stretches, ARE, INS and CRS sequence elements and cryptic splice donor and acceptor sites.

These mutations did not change the amino acid sequence of the invention. When this sequence was expressed in HEK-293 cells and Xenopus oocytes (See examples below) it was found to be efficiently expressed and to result in a functional ion channel that responded specifically to coolant compounds.

### 1. Exemplary Materials and Methods Used for Calcium Imaging Experiments

HEK-293 cells (about 50-70% confluency) contained in 10 cm dishes are transfected with 5 μg of TRPM8 DNA and pcDNA3 and 2 μg of RFP plasmids using TransH293.

After 24 hours, the cells are split into 384-well plates at ˜50,000 cells/well.

At 48 hours post-transfection the cells are loaded with 4 μM Fluo-3-AM 3 AM in HBSS for 30 minutes at 37° C.

The cells are then washed twice within HBSS containing 2.5 mM Probenicid and returned to 37° C. for 15 minutes.

Compound plates are prepared in HBSS at twice the final concentration and kept at 37° C. to insure that rTRPM8 is not activated by a decrease in ambient temperature during stimulation (TRPM8 is activated by temperatures<22° C.).

### Materials and Methods Used for Selection of Stable Clones

HEK-293 cells transfected with TRPM8 nucleic acid sequence containing plasmid that comprises a neo marker and stable cell clones are selected using neomycin.

Screened clones are screened using calcium imaging for a (−) menthol response on FLIPR.

Clones that exhibit optimal menthol response are selected based on TRPM8 activation detected by use of calcium imaging.

### 2. Methods and Materials Used for Patch Clamp Electrophysiological Assays

Xenopus oocytes are microinjected with a TRPM8 nucleic acid sequence according to the invention.

The microinjected oocytes are voltage-clamped at around 60 mV using the OpusXpress 600A one day post-injection and treated with either buffer (control) or a potential or known TRPM8 modulator contained in same buffer at a fixed concentration or over a range of different concentrations (dose-escalation).

The current is measured for said buffer-treated or putative TRPM8 modulator-treated oocytes over a specific time period. Additionally, the current response is measured for oocytes treated with the same buffer which are not injected with a TRPM8 nucleic acid sequence (negative control).

The current response for said oocytes is compared in order to determine the effect (if any) of said potential TRPM8 modulator compound on TRPM8 activity and whether said effect is dose-specific.

### Example 2

### Activation of Rat TRPM8 Expressed In HEK293 Cells

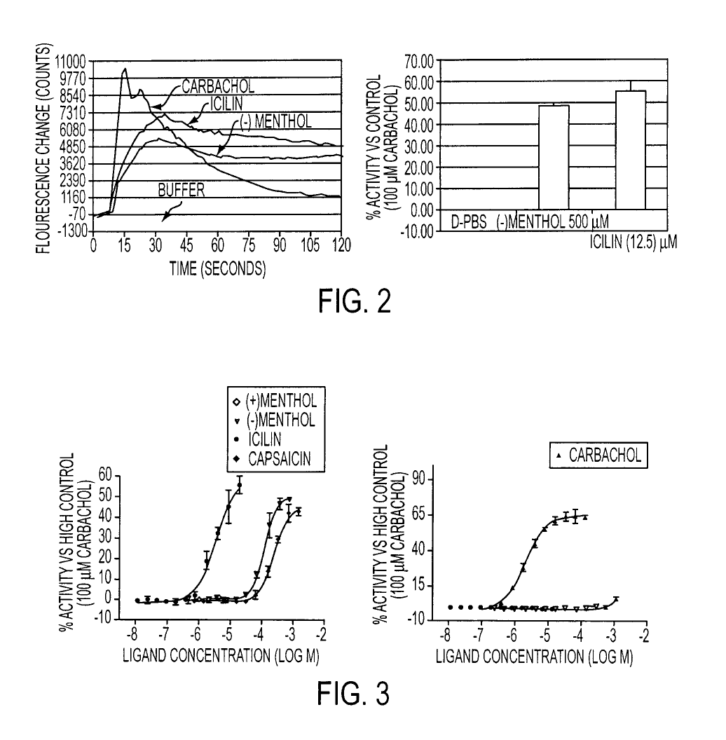

HEK293 cells are transfected with a plasmid encoding the rat TRPM8 cDNA (in pcDNA3.1) and are seeded into 384-well plates. 48 hours later, cells are loaded with Fluo-3-AM. Cells are then stimulated with various stimuli as shown in FIG. 2 and fluorescence intensity in each well measured using a Fluorimetric Imaging Plate Reader (FLIPR). In these experiments, carbachol stimulation of endogenously expressed M1 receptors was used as a reference stimulus. The results in FIG. 2 show that the tested coolant compounds specifically activate the rat TRPM8 ion channel.

### Example 3

### Rank Order of Cooling Agents that Activate Rat TRMP8

HEK293 cells transfected with a plasmid encoding rat TRPM8 cDNA contained in pcDNA3.1 were seeded into 384-well plates. 48 hours later, these cells were loaded with Fluo-3-AM. Cells were then stimulated with the stimuli shown in FIG. 3 and fluorescence intensity in each well measured using a Fluorimetric Imaging Plate Reader (FLIPR). Carbachol stimulation of endogenously expressed M1 receptors was again used as a reference stimulus. The Panel on the right in FIG. 3 shows that cells transfected with a control plasmid (RFP) respond only to Carbachol stimulation. The results in the left Panel of FIG. 3 shows that rat TRPM8 responds to the coolant compounds shown therein.

### Example 4

### Synergistic Activation of TRPM8 with Cool Temperatures and Cooling Agents

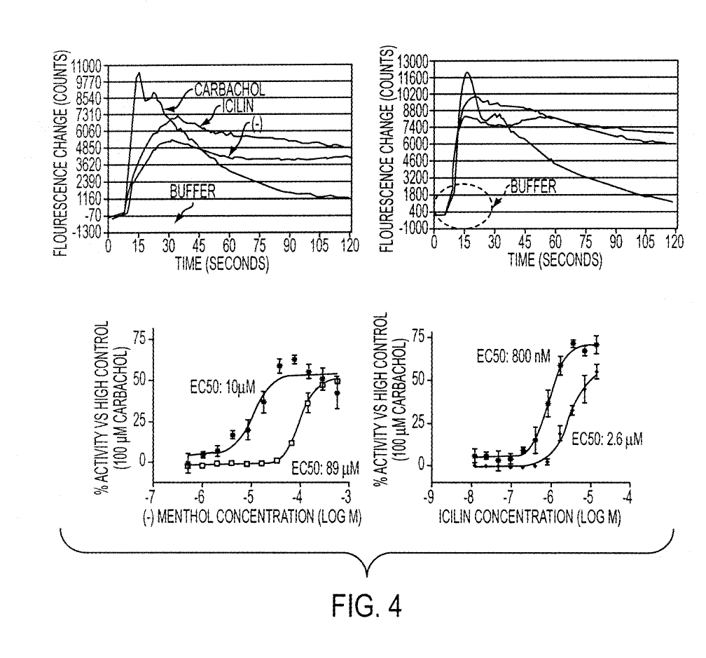

As shown in FIG. 4, cool temperatures activate TRPM8 in HEK293 and exhibit a synergistic effect in conjunction with cooling agents. HEK293 cells which were transfected with a plasmid encoding rat TRPM8 cDNA in pcDNA3.1 were again seeded into 384-well plates. 48 hours later, these transfected cells were loaded with Fluo-3-AM. Cells were then stimulated with the stimuli shown in FIG. 4 and fluorescence intensity in each cell measured using a Fluorimetric Imaging Plate Reader (FLIPR). The results in the top right panel of FIG. 4 show that the addition of cold buffer as a stimulus is sufficient to induce TRPM8 activation. The results in the bottom panels of FIG. 4 show that chilled (--) menthol and chilled icilin are more potent than warmer menthol and icilin activating TRPM8.

### Example 5

### Menthol and Icilin Activate Rat TRPM8 Expressed in Oocytes

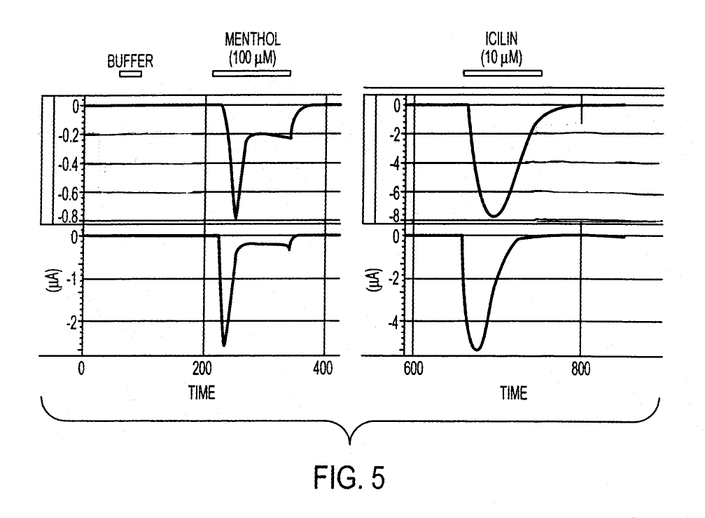

In this experiment an electrophysiological assay was conducted using oocytes that express rat TRPM8. Specifically, oocytes were microinjected with 10 ng rat TRPM8 cRNA and were voltage-clamped at ˜60 mV using the OpusXpress 600A one day post-injection and treated with buffer and menthol (left traces) or icilin (right traces). Two oocytes that responded to the indicated treatments are shown in FIG. 5. These results indicated that menthol-induced currents partially-desensitize (currents peak and decline to a steady-state in the continued presence of agonist) whereas icilin-induced currents completely desensitize (currents reference to control levels in the continued presence of agonist). By contrast, currents were not affected by treatment with the buffer.

### Example 6

### Specific Activation of Rat TRPM8 Oocytes by Coolants

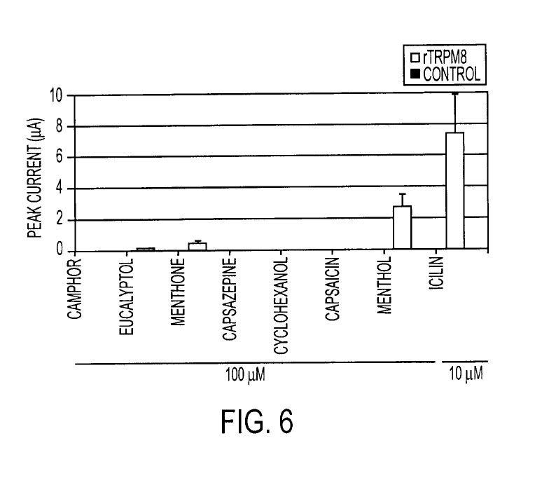

In this experiment, it was shown that menthol and icilin specifically activate rat TRPM8 expressed in oocytes. Oocytes were microinjected with 10 ng rat TRPM8 cDNA and voltage-clamped at ˜60 mV using the OpusXpress 6000A one day post-injection and then treated with the compounds shown in the FIG. 6. In the Figure, peak agonist-induced currents are summarized for 4-6 independent oocytes. The results of these experiments revealed that menthol and icilin induced large peak currents whereas eucalyptol and methane only induced small peak currents at the indicated compounds concentrations. By contrast, no responses were observed in control oocytes that do not express rat TRPM8 (control; uninjected oocytes). Thus, the results in FIG. 6 show that menthol and icilin specifically activate rat TRPM8 expressed in oocytes.

### Example 7

### Menthol Activation of Rat TRPM8 in Oocytes Expressing Rat TRPM8

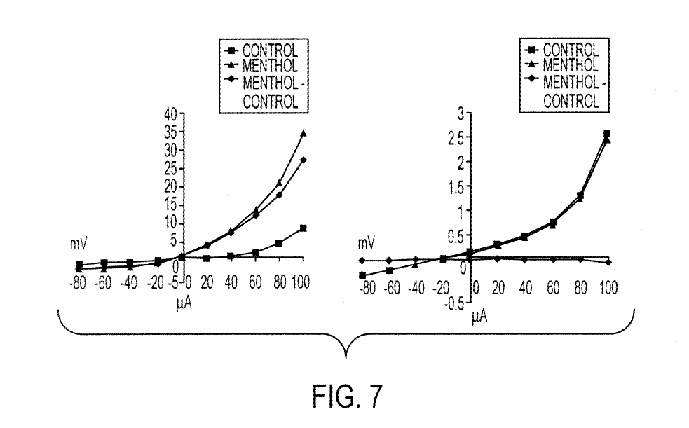

Experiments were conducted that revealed that menthol current/voltage (I/V) curves display outward rectification in oocytes that express rat TRPM8. In these experiments, oocytes were again injected with 2 ng rat TRPM8 cRNA (as shown in left panel of FIG. 7) or uninjected (right panel) and currents were measured from ˜80 mV to 100 mV (in 20 mV increments) in the presence of buffer (control; green curves) or 100 μM menthol (red curves) four days post-injection. The blue curves in FIG. 7 depict menthol-specific currents obtained by subtracting control (green) from menthol (red) curves. The results in FIG. 7 revealed that menthol-specific currents exhibit outward rectification (currents are larger or positive voltages in comparison to negative voltages) whereas no menthol-specific currents are observed in control (uninjected) cells.

### Example 8

### Menthol Dose-Response Curve in Rat TRPM8 Expressing Oocytes

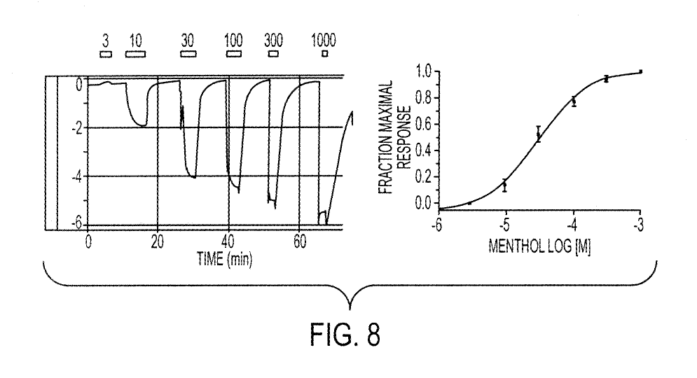

FIG. 8 contains the results of experiments measuring dose-response for menthol in rat TRPM8 expressing oocytes. In this experiment, oocytes were again microinjected with 10 ng rat TRPM8 cRNA, voltage-clamped at ˜60 mV and currents measured 2-3 days post-injection. The results in the left panel of FIG. 8 depict a representative experiment in an oocytes treated with increasing concentrations of menthol from 3 μM to 1000 μM. The results in the right panel of FIG. 8 depict summarized data where each point corresponds to data from 3-6 independent oocytes. As shown in the figure, the EC₅₀ value for menthol was 29.6 μM at 19.5° C. This value is close to the reported EC₅₀ for menthol in oocytes (67 μM at 22-24° C., McKemy et al. Nature 416:52-58 (2002)), confirming the validity of the experimental results.

### Example 9

### Activation of Rat TRPM8 by Cool Temperatures

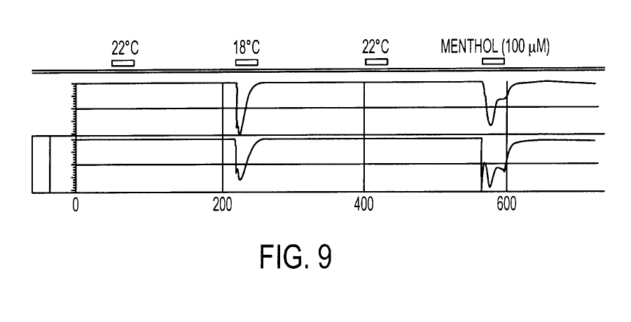

FIG. 9 contains the results of an experiment showing that cool temperatures activate rat TRPM8 expressed in oocytes. In this experiment, oocytes were again microinjected with 5 ng rat TRPM8 cRNA, voltage-clamped at −60 mV and currents recorded two days post-injection using the OpusXpress 6000A. As shown in the Figure, application of room temperature buffer (22° C.) had no effect on measured currents, whereas application of buffer cooled to 18° C. activated rat TRPM8 to a similar extent as menthol at room temperature. Two oocytes responding to the indicated temperature treatment are shown in FIG. 9.

### Example 10

### Effects of Various Coolants on HEK293 Clones Stably Expressing Rat TRPM8

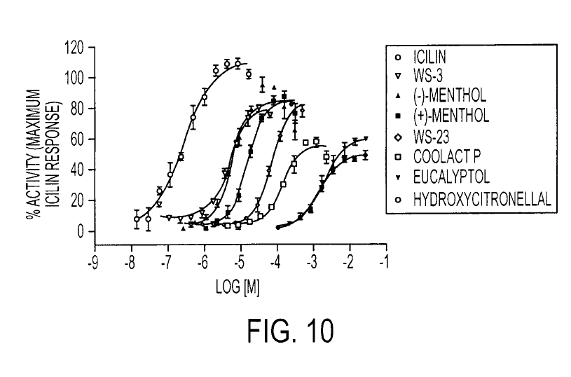

FIG. 10 shows the properties of a HEK293 clone stably expressing rat TRPM8. HEK293 cells were again seeded into 384-well plates and 48 hours later cells were loaded with Fluo-3-AM dye. Cells were then stimulated with the stimuli shown in FIG. 10 and fluorescence intensity in each cell measured using a Fluorimetric Imaging Plate Reader (FLIPR). The results contained in FIG. 10 shows that these indicated cooling agents specifically activate the stable clone with the order of potency and cooling strength reported therein.

### Example 11

### Identification of a Proprietary Compound that Activates Rat TRPM8 More Potently than Menthol

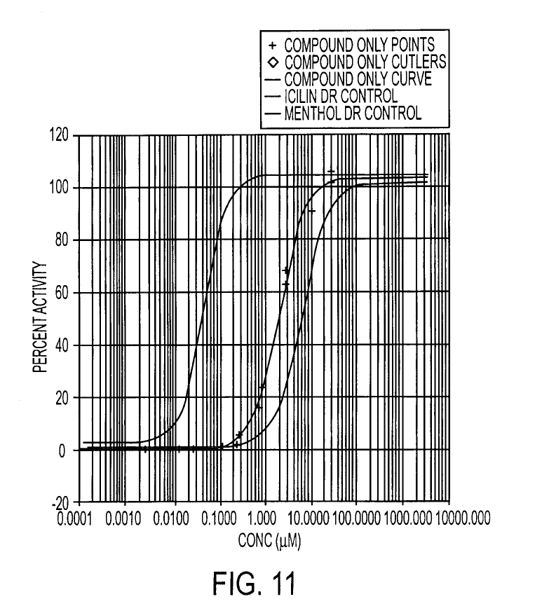

Fluorimetric Imaging experiments were conducted as described in Example 9 also using the stable HEK293 clone described therein. Specifically, 19,000 compounds were screened against this clone and positive hits were subsequently analyzed by close-response. These experiments identified a proprietary compound (SID-2346448) that was reproducibly 2-3 times more potent than (--) menthol at activating rat TRPM8. These results are contained in FIG. 11.

### Example 12

### Identification of a Second Proprietary a Compound that Activates Rat TRPM8 More Potently than Menthol

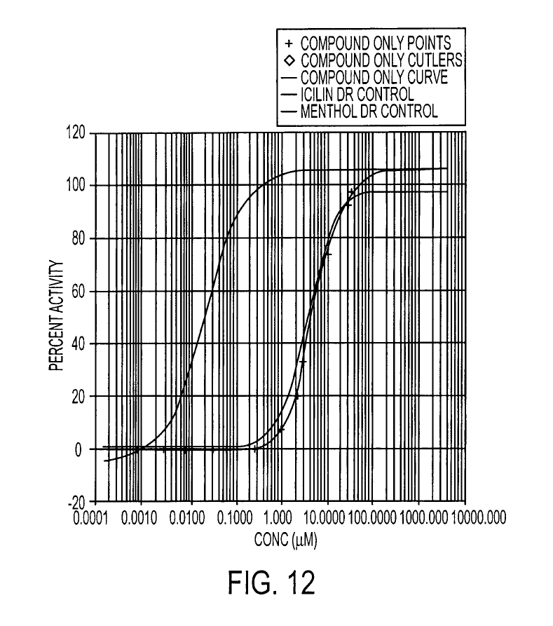

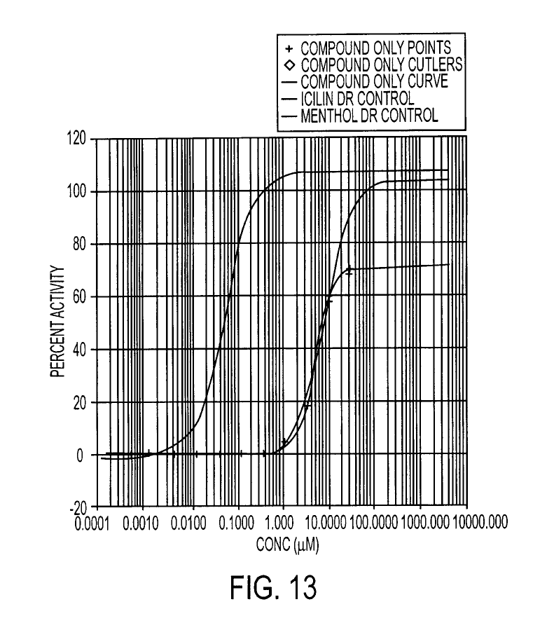

A fluorimetric calcium imaging experiment was conducted as described in Example 9 using the stable HEK293 clone described therein. A total of 19,000 compounds were again screened against clone #48. The positive hits were subsequently analyzed by close-response. These results revealed that a second proprietary compound (SID 576583) was reproducibly as potent as (--) menthol at activating rat TRPM8. These results are contained in FIG. 12.

### Example 13

### Identification of a Third Proprietary Compound that Activates Rat TRPM8 More Potently than Menthol

Fluorimetric calcium imaging experiments were again conducted using the stable HEK293 clone as described in Example 10. A total of 19,000 compounds were screened against this clone (clone #48). The positive hits were then subsequently analyzed by dose-response. These experiments revealed the identity of a third proprietary compound (SID 3498787), that reproducibly is as potent as (--) menthol at activating rat TRPM8.

### Example 14

### Properties of Human TRPM8 Expressed in HEK293 Cells

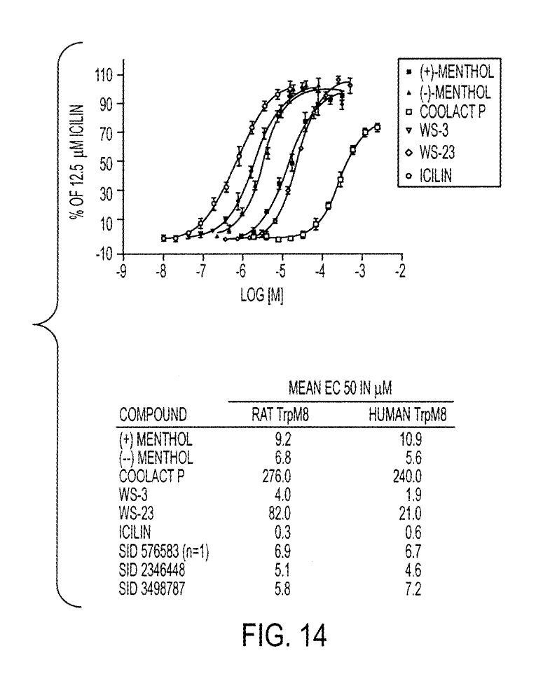

FIG. 14 contains the results of an experiment studying the properties of human TRPM8 expressed in HEK293 cells. In these experiments, HEK293 cells transfected with a plasmid encoding the modified human TRPM8 cDNA in FIG. 1 were seeded into 384-well plates. 48 hours later, these cells were loaded with Fluo-3-AM. These cells loaded with Fluo-3-AM were then stimulated with the stimuli shown in FIG. 14 and fluorescence intensity in each well measured using a Fluorimetric Imaging Plate Reader (FLIPR). The indicated cooling agents activate TRPM8 according to the reported rank order of potency and cooling strength. The results in the table contained in FIG. 14 further compare EC50s obtained with rat TRPM8 and human TRPM8-expressing cells. It can be seen that these EC50 values are consistent with one another in these cells for the different coolants tested.

### Example 15

### Properties of a HEK-293 Clone Expressing Human TRPM8 According to the Invention (SEQ ID NO:2)

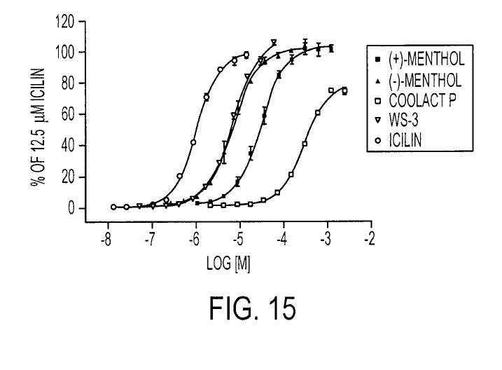

The experiment compared the properties of a HEK-293 clone expressing an optimized hTRPM8 nucleic acid sequence according to the invention (“hTRPM8 opt” or SEQ ID NO: 2). Particularly, these cells were again seeded into 384-well plates and 48 hours later were loaded with a fluorescent dye (Fluo-3 AM). The resultant loaded cells were then stimulated with the stimulants indicated in FIG. 15 and the fluorescence intensity for each cell measured using a Fluorimeter Imaging Plate Reader (FLIPR). As may be seen from the results contained in FIG. 15, the tested known cooling agents were observed to activate the stable hTRPM8 expressing clone with the rank order of potency and cooling strength activity reported therein.

### Example 16

### Potency of Several Putative Identified in Inventive Screens

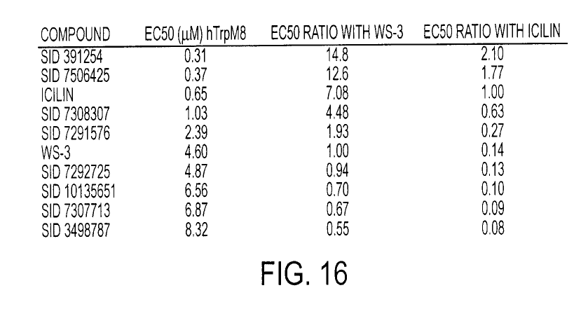

A screen was performed against 15 thousand compounds on clone #71 (same clone as prior example). The “hits” were then subsequently evaluated by dose-response analysis. These results which are summarized in the table in FIG. 16 revealed that SID 391254 and SID 7506425 were reproducibly as potent as icilin, a known coolant, at activating human TRPM8. Also, other compounds, SID 7308307, SID 7291576 and SID 7292725, were reproducibly as potent as WS-3, another known coolant, at activating rat TRPM8. Further, the rest of the hits, SID 10135651, SID 7307713 and SID 3498787 were as potent as (−) menthol at activating human TRPM8.

### Example 17

### Cooling Effect of Putative Coolant Compound (SID 391254) in Human Taste Tests

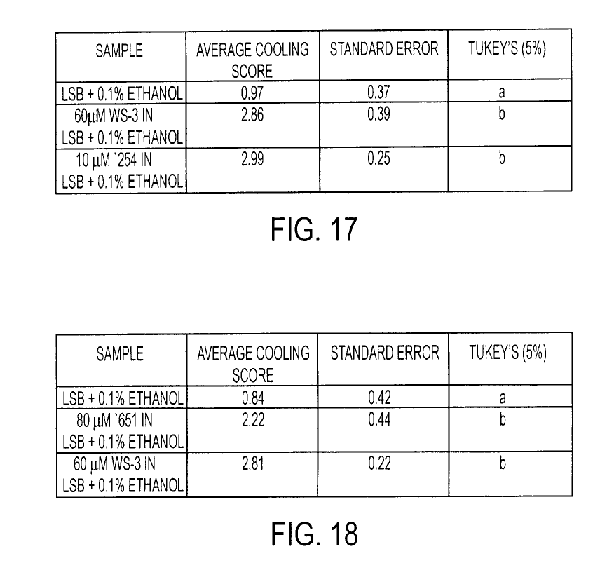

In this experiment the cooling effect of a putative coolant, SID 391254, identified using the subject assays was analyzed in human taste tests. Particularly, the cooling intensity for three test samples was tested in five human volunteer panelists in two trials. The results of these trials contained in FIG. 17 revealed significant calculated differences using Tukey's HSD (5% risk level). In this experiment, samples with the same Tukey's lettering were not significantly different from one another. The tests were conducted in booths with the data recorded using Compusense software. Additionally, these experiments further included the administration of WS-3, a known coolant (positive control).

For both samples containing the known or the putative cooling compound (WS-3 and SID 391254 respectively), these compounds were contained in low sodium buffer (LSB) and 0.1% ethanol. As reported in the Figure, the samples containing the known or putative coolant reported substantially higher cooling intensity than the negative control (LSB and 0.1% ethanol). These results are consistent with the fact that LSB and ethanol exhibit no known coolant effect. Also, it was found that the putative coolant compound 391254 is actually more potent than WS-3 since it was used at ⅙ molar concentration of WS-3 and produced the same effect.

### Example 18

### Cooling Effect of Second Putative Cooling Compound (SID 10135651) in Human Taste Tests

This experiment compared the cooling effect of another putative coolant compound (SID 10135651) identified using the described assays. This compound was again compared in human taste tests to a known coolant WS-3 and the same negative control sample (LSB containing 0.1% ethanol). In this experiment the average cooling intensity was again compared for the three samples identified in FIG. 18 in five human volunteer in two trials. As with the prior example, significant differences between the known and putative coolant compound vis-à-vis the control were calculated using Tukey's HSD (5% risk level). Also, samples with the same Tukey's lettering were not significantly different from each other. These tests were again conducted in booths with the data recorded using Compusense software. These comparisons revealed that the sample containing the SID 10135651 compound and WS-3 exhibited substantially higher cooling intensity than the control samples. The results of this experiment further revealed that the SID 10135651 compound sample exhibited about the same cooling intensity as the WS-3 sample.

### Example 19

### Cooling Effect of Another Putative Coolant Compound (SID 7292725) in Human Taste Tests

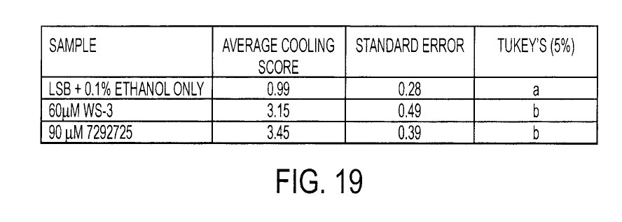

The coolant effect of another putative cooling compound (SID 7292725) identified using the subject screening assays was tested in human taste tests. Again cooling intensity scores were determined based on results in 5 human taste panelists in two trials. Significant differences were again calculated using Tukey's HSD (5% risk level). [Tukey's (5% equaled 1.279]. Similarly, the samples with the same Tukey's lettering were not significantly different from each other. All of the samples were again prepared in LSB containing 0.1% ethanol. Further, WS-3 was again used as the known comparison coolant compound. As shown in FIG. 19, the samples containing the known and putative coolant compounds elicited higher cooling intensity than the negative control (LSB and 0.1% ethanol). Also, no significant differences in the cooling intensity between the WS-3 and SID 7292725 samples were observed.

[image]

[image]

[image]

We claim:

1. A modified human TRPM8 nucleic acid sequence that: (i) comprises a nucleic acid sequence that is modified relative to the wild-type TRPM8 nucleic acid sequence contained in SEQ ID NO:2 or another wild-type TRPM8 nucleic acid sequence at least by mutations that remove one or more of the following: (1) TATA-boxes, (2) chi-sites, (3) ribosomal entry sites, (4) ARE, INS, or CRS sequence elements, and (5) cryptic splice donor and acceptor sites, and (ii) is expressed in human cells as an active ion channel which possesses substantially the same ligand binding and functional activity as the polypeptide encoded by the nucleic acid sequence contained in SEQ ID NO:2.

2. The modified nucleic acid sequence of claim 1 which is operably linked to a promoter.

3. The modified nucleic acid sequence of claim 2 wherein the promoter is a regulatable or constitutive promoter.

4. The modified nucleic acid sequence of claim 1 which contains at least 100 silent sequence modifications.

5. The modified nucleic acid sequence of claim 1 which contains at least 200 silent modifications.

6. The modified nucleic acid sequence of claim 1 which contains at least 300 silent modifications.

7. The modified nucleic acid sequence of claim 1 which contains at least 400 silent modifications.

8. The modified nucleic acid sequence of claim 1 which contains at least 500 silent modifications.

9. The modified nucleic acid sequence of claim 1 which contains at least 600 silent modifications.

10. The modified nucleic acid sequence of any of claims 4-9 wherein said silent modifications are selected from those contained in SEQ ID NO: 2 as compared to the unmodified nucleic acid sequence contained in SEQ ID NO:1.

11. The modified nucleic acid sequence of claim 1 which possesses at least 95-99% sequence identity to the TRPM8 nucleic acid sequence contained in SEQ ID NO:2.

12. Th modified nucleic acid sequence of claim 1 wherein said nucleic acid sequence possesses the nucleic acid sequence contained in SEQ ID NO:2.

13. The modified sequence of claim 12 which is operably linked to a regulatable or constitutive promoter.

14. The modified sequence of any one of claims 1-9 or 11-13 which is contained on a plasmid.

15. A primate cell or oocyte transfected, transformed or microinjected with a nucleic acid sequence according to any one of claims 1-9 or 11-13.

16. A primate cell or oocyte transfected, transformed or microinjected with a nucleic acid sequence according to claim 12.

17. The cell of claim 15 which is a human cell.

18. The cell of claim 16 which is a human cell.

19. The cell of claim 15 which is a HEK-293 cell, African Green Monkey cell, or Cos cell or CHO cells.

20. The cell of claim 16 which is a HEK-293 cell or a Cos cell or a CHO cell.

21. A method for identifying compounds that modulate the activity of a human TRPM8 ion channel which is encoded by a modified human TRPM8 nucleic acid sequence comprising:
(i) obtaining a cell that expresses a modified human TRPM8 nucleic acid sequence, wherein such modified human TRPM8 nucleic acid sequence is modified relative to the human TRPM8 nucleic acid sequence contained in SEQ ID NO: 2 at least by the introduction of mutations selected from the group consisting of removal of putative (1) TATA-boxes, (2) chi-sites, (3) ribosomal entry sites, (4) ARE, INS or CRS sequence elements, and (5) cryptic splice donor and acceptor sites;
(ii) contacting said cell expressing said modified human TRPM8 nucleic acid sequence with a putative modulator of the human TRPM8 ion channel; and
(iii) identifying whether said compound modulates the activity of the human TRPM8 ion channel encoded by said modified human TRPM8 nucleic acid sequence.

22. The method of claim 21 wherein the cell that expresses said nucleic acid sequence is a mammalian cell.

23. The method of claim 21 wherein the cell that expresses said nucleic acid sequence is a human cell.

24. The method of claim 21 wherein the cell that expresses said nucleic acid sequence is selected from the group consisting of HEK-293, BHK, CHO, COS, monkey L cell, African green monkey kidney cell, Ltk-cell and an oocyte.

25. The method of claim 21 wherein said nucleic acid sequence possesses from about 80-85% sequence identity to the human TRPM8 nucleic acid sequence contained in SEQ ID NO:1.

26. The method of claim 25 wherein said nucleic acid sequence possesses the nucleic acid sequence contained in SEQ ID NO:2.

27. The method of claim 21 wherein the modified TRPM8 nucleic acid sequence contains at least 100-200 silent mutations.

28. The method of claim 21 wherein the modified TRPM8 nucleic acid sequence contains at least 300-400 silent mutations.

29. The method of claim 21 wherein said modified TRPM8 nucleic acid sequence contains at least 500 silent mutations.

30. The method of claim 21 wherein said modified TRPM8 nucleic acid sequence contains at least 550 silent mutations.

31. The method of any one of claims 27-30 wherein said silent mutations are selected from the 601 silent mutations contained in SEQ ID NO:2.

32. The method of claim 21 which further comprises identifying whether a compound identified as a human TRPM8 modulator in said assay method is further evaluated in human taste tests or human skin contact (topical) tests to assess whether it elicits a cooling effect or enhances the cooling effect of another coolant.

33. The method of claim 21 wherein human TRPM8 activity is assayed by detecting whether said compound affects concentrations of intracellular calcium.

34. The method of claim 21 wherein human TRPM8 activity is assayed by detecting whether said compound affects concentrations of intracellular sodium.

35. The method of claim 21 wherein said assay comprises a step whereby the human TRPM8 encoded by said nucleic acid sequence is stimulated by cold temperature or a coolant compound known to activate human TRPM8.

36. The method of claim 34 wherein said compound known to activate human TRPM8 is menthol, icilin or a derivative thereof.

37. The method of claim 21 wherein TRPM8 activity is monitored using a fluorescent calcium-sensitive dye.

38. The method of claim 21 wherein TRPM8 activity is monitored using a sodium-sensitive dye.

39. The method of claim 21 wherein TRPM8 activity is monitored using a membrane potential dye.

40. The method of claim 37 wherein said dye is Fura2, Fluo3 or Fluo4.

41. The method of claim 39 herein said membrane potential dye is selected from the group consisting of Molecular Devices Membrane Potential Kit (cat#R8034), Di-4-ANEPPS (pyridinium, 4-(2-(6-(dibutylamino)-2-naphthalen-yl)ethenyl)-1-(3-sulfopropyl))-hydroxide, inner salt, DiSBACC4(2)(bis-(1,2-dibabituric acid)-trimethine oxanol), DiSBAC4(3)(bis-(1,3-dibarbituric acid)-trimethine oxanol), Cc-2-DMPE (Pacific Blue 1,2-dietradecanoyl-sn-glycerol-3-phosphoeyhanolamine,triethylammonium salt) and SBFI-AM (1,3-benzenedicarboxylic acid, 4,4-[1,4,10-trioxa-7,13-diazacyclopentadecane-7,13-diylbis(5-methoxy-6,1-2-benzofurandiyl)]bis-,tetrakis[(acetyloxy)methyl]ester (Molecular Probes).

42. The method of claim 38 wherein said sodium sensitive dye is sodium green tetraacetate (Molecular Probes) or Na-Sensitive Dye Kit (Molecular Devices).

43. The method of claim 21 wherein said cell transiently expresses said modified human TRPM8 nucleic acid sequence.

44. The method of claim 21 wherein said cell stably expresses said modified human TRPM8 nucleic acid sequence.

45. The method of claim 21 wherein TRPM8 activity is monitored by an ion flux assay.

46. The method of claim 45 which uses a radiolabel to detect TRPM8 flux.

47. The flux assay of claim 45 which uses atomic absorption spectroscopy to detect ion flux.

48. The method of claim 21 wherein said modified human TRPM8 nucleic acid sequence is operably linked to a regulatable promoter.

49. The method of claim 21 wherein said modified human TRPM8 nucleic acid sequence is operably linked to a constitutive promoter.

50. The method of claim 21 which is a high throughput compound screening assay.

51. The method of claim 21 wherein the effect of said screened compound on the activity of said human TRPM8 is assayed electrophysiologically.

52. The method of claim 51 which comprises using patch clamping.

53. The method of claim 51 which comprises two electrodes voltage clamping.

54. The method of claim 51 which uses an automatic voltage or current recording instrument.

55. The method of claim 21 wherein said instrument is a fluorescence plate reader (FLIPR) or is a voltage imaging plate reader (VIPR).

56. The method of claim 54 wherein said instrument is an OpusXpress or IonWorks.

57. The method of claim 21 which screens for compounds that are at least equipotent with menthol or icilin at activating rat or human TRPM8.

58. A test kit for identifying a human TRPM8 modulator which comprises:
(i) a test cell that stably or transiently expresses a modified human TRPM8 nucleic acid sequence that encodes a human TRPM8 polypeptide which nucleic acid sequence is modified relative to the human TRPM8 nucleic acid sequence contained in SEQ ID NO: 1 at least by the introduction of mutations selected from the group consisting of removal of putative (1) TATA-boxes, (2) chi-sites, (3) ribosomal entry sites, (4) ARE, INS or CRS sequence elements, and (5) cryptic splice donor and acceptor sites; and
(ii) a detection system for detecting whether a compound modulates the activity of human TRPM8.

59. The test kit of claim 58 wherein said cell expresses the nucleic acid sequence contained in SEQ ID NO: 2.

60. The test kit of claim 58 wherein sad modified TRPM8 nucleic acid sequence contains at least 200-400 silent mutations.

61. The test kit of claim 58 wherein said modified TRPM8 nucleic acid sequences contains at least 400-600 silent mutations.

62. The test kit of claim 58 wherein said modified TRPM8 nucleic acid sequence contains at least 500-600 silent mutations.

63. The test kit of any one of claims 60-62 wherein said silent mutations are selected from the 601 silent mutations contained in SEQ ID NO:2.

64. The test kit of claim 58 wherein the detection system includes a means for detecting intracellular calcium or sodium or voltage.

65. The test kit of claim 58 wherein the detection system includes a calcium sensitive or sodium sensitive dye.

66. The test kit of claim 58 wherein the detection system comprises a patch clamp or two electrode clamp electrophysiological detection system.

67. The test kit of claim 58 wherein said test cell transiently expresses said nucleic acid sequence.

68. The test kit of claim 58 wherein said test cell stably expresses said nucleic acid sequence.

69. The test kit of claim 59 wherein the cells are human cells.

70. The test kit of claim 49 wherein said cells are HEK-293 cells.
